(19) United States
(12) Patent Application Publication
(10) Pub. No.: US 2024/0123255 A1
(43) Pub. Date: Apr. 18, 2024

(54) PERCUTANEOUS PHOTOBIOMODULATION

(71) Applicant: Case Western Reserve University, Cleveland, OH (US)

(72) Inventors: Michael MOFFITT, Cleveland, OH (US); Michael JENKINS, Cleveland, OH (US)

(21) Appl. No.: 18/546,680

(22) PCT Filed: Feb. 21, 2022
(86) PCT No.: PCT/US2022/017171

§ 371 Date: Aug. 16, 2023

Related U.S. Application Data
(60) Provisional application No. 63/151,074, filed on Feb. 19, 2021.

(51) Int. Cl. A61N 5/06; A61N 5/067

(52) U.S. Cl. CPC A61N 5/0622; A61N 5/067; A61N 2005/0612; A61N 2005/0626; A61N 2005/063; A61N 2005/0651

(57) ABSTRACT

A system for percutaneous photobiomodulation (PBM) that can deliver light that is configured outside the body directly to target tissue is described. The system can be used to treat both chronic and temporary conditions with PBM. The system includes a light source and a controller that includes a memory storing a predefined dosing requirement and a processor to access the memory and signal the light source to generate a light signal for PBM of a target area within a patients body based on the predefined dosing requirement. In some instances, the system also includes a port to deliver the light signal received from the light source to the target area. The light source and the port can be connected by an attachment that can transmit the light signal from the light source to the port.

### CROSS-REFERENCE TO RELATED APPLICATIONS

This application claims the benefit of U.S. Provisional Application Ser. No. 63/151,074, filed 19 Feb. 2021, entitled “PERCUTANEOUS PHOTOBIOMODULATION”, the entirety of which is incorporated by reference for all purposes.

### TECHNICAL FIELD

The present disclosure relates generally to photobiomodulation (PBM) and, more specifically, to a system that can be used for chronic or temporary percutaneous PBM (e.g., port-based PBM).

### BACKGROUND

Generally, photobiomodulation (PBM) describes the delivery of light with proper wavelengths to a patient at a specific dosing scheme to achieve a desired non-thermal response at a target location. Typical wavelengths used for PBM are in the range of 600 nm to 1200 nm. It should be noted that longer wavelengths greater than 1400 nm can create a thermal effect. Based on the wavelengths used and the dosages, PBM theoretically can be used to achieve varied responses at different target locations in a patient's body. Some of these varied responses, like nerve block, anti-inflammation (e.g., by activating anti-inflammatory microglia), anti-neurodegeneration (e.g., by overcoming cellular oxidative stress), anti-fibrotic responses in pathological fibrosis, improved cellular function (e.g., by improved cellular respiration), and the like, can be used to treat chronic conditions. However, PBM has only achieved these varied responses to modest effects, likely due to delivery constraints of the traditional transcutaneous delivery of PBM.

With traditional transcutaneous delivery, the light must travel through the patient's skin and other tissue layers of the patient's body to reach the target location, making the PBM source require greater power for some measure of effective light to reach the target location, often outside of imposing power constraints for safe delivery of the PBM. Additionally, the skin and tissue layers can absorb the light of the PBM. It is well recognized that a greater effect may be achieved with lower power requirements if the light were to be applied directly to the target location. As such, single-use percutaneous systems and fully implantable systems have been investigated to accomplish direct application of PBM to the target location. However, single use percutaneous systems are not well-suited to treat chronic disease, and fully implantable systems are costly with power limitations that may not support delivery of PBM at the required dose.

### SUMMARY

As an alternative to traditional transcutaneous systems, investigational percutaneous single-use systems, and fully implantable systems, the present disclosure relates to a system for percutaneous photobiomodulation (PBM) (which may also include multi-use systems). One example of a percutaneous solution is a port-based system. The port-based system can be used to treat both chronic and temporary conditions with PBM by delivering light directly to the target tissue and that is configured outside the body.

In an aspect, the present disclosure can include a system that can be used to deliver PBM to a target area in a patient's body. The system can include a light source and a controller that includes a memory storing a predefined dosing requirement and a processor configured to access the memory and signal the light source to generate a light signal for PBM of a target area within a patient's body based on the predefined dosing requirement. The system can also include a port to deliver the light signal received from the light source to the target area. The light source and the port can be connected by an attachment that is configured to transmit the light signal from the light source to the port. The port and the light source can be disconnected and connected as needed.

In another aspect, the present disclosure can include a method for delivering PBM to a target area in a patient's body. The method includes accessing, by a controller comprising a processor, a predefined dosing requirement; signaling, by the controller, a light source to generate a light signal for photobiomodulation of a target area within a patient's body based on the predefined dosing requirement; and delivering the light signal received from the light source to the target area through a port. The light source and the port are connected/disconnected through an attachment device.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a diagram showing an example of a port-based system that can be used to deliver percutaneous photobiomodulation (PBM) in accordance with an aspect of the present disclosure;

FIG. 2 is a diagram showing an example extension of the port-based system shown in FIG. 1 to include an external programmer;

FIGS. 3-5 are diagrams showing different example systems for percutaneous PBM;

FIGS. 6-8 are diagrams showing different example port-based systems for delivering PBM into the brain;

FIG. 9 is a process flow diagram illustrating a method for using a percutaneous system for PBM in accordance with another aspect of the present disclosure;

FIGS. 10 and 11 show example configurations of a percutaneous treatment for headache syndromes; and

FIG. 12 shows an example configuration of a percutaneous treatment for delivery of light to an affected lung.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “photobiomodulation (PBM)” can refer to a form of light therapy based on the delivery of light with one or more proper wavelengths to a patient at a specific dosing scheme to achieve a desired response (or effect) at a target area. PBM utilizes non-ionizing light sources, including lasers, light emitting diodes, and/or broadband light. In some examples, the light can have a wavelength between 250 and 1600 nm. However, as an example, the wavelength can be in the visible range (e.g., 400 nm-700 nm) and/or near-infrared range (e.g., 700 nm-1100 nm) of the electromagnetic spectrum.

As used herein, the term “percutaneous” can refer to something that is made, done, or effected through the skin from extracorporeal to intracorporeal. For example, a port can be one way to span the skin and provide an entrance from the extracorporeal region to the intracorporeal region. However, other devices can be used for percutaneous entry into the body (e.g., a light pipe itself without requiring a port).

As used herein, the term “port” can refer to a device including an opening, passage, or channel through which something extracorporeal (e.g., light, pharmaceutical, electrical, mechanical, etc.) can be introduced intracorporeally (into the body).

As used herein, the term “extracorporeal” can refer to something being outside a subject or patient's body (or, in other words, outside the skin).

As used herein, the term “intracorporeal” can refer to something being within the body (or, in other words, under the skin).

As used herein, the terms “target area” and “target location” can refer to a portion of a subject's body in need of PBM.

As used herein, the term “dosing requirement” can refer to one or more characteristics of a dose for treating a medical condition.

As used herein, the terms “subject” and “patient” can be used interchangeably and refer to any warm-blooded organism including, but not limited to, a human being, a pig, a rat, a mouse, a dog, a cat, a goat, a sheep, a horse, a non-human primate, a rabbit, a cow, etc.

### II. Overview

Photobiomodulation (PBM) can be used to achieve varied responses at different target locations in a patient's body to treat chronic conditions. However, due to constraints inherent to traditional transcutaneous delivery, PBM has only achieved these varied responses to modest effects. A greater effect may be achieved if the light were to be applied directly to the target location, but single-use percutaneous systems cannot be used chronically and fully implantable systems are costly with power limitations that may not support delivery of PBM at the required dose. Port-based systems (and other percutaneous systems) overcome the aforementioned problems and limitations of traditional PBM delivery routes.

The present disclosure describes a port-based system (and other percutaneous systems) that can provide chronic or temporary PBM to many target areas, delivering one or more doses of light to the patient per day according to a predefined dosing requirement. The port-based system (and other percutaneous systems) is advantageous over traditional transcutaneous delivery because the port-based system can deliver light to the target tissue directly, minimizing absorption by intervening tissue layers (this absorption can increase the power requirements and at the same time limit the amount of power that can be safely delivered). Additionally, the port-based system (and other percutaneous systems) can be used to treat chronic illness, healing, and recovery that cannot be accomplished by single use systems. Moreover, the port-based system (and other percutaneous systems) is lower cost, more MRI compatible, and more adaptable (in terms of wavelengths, doses, delivery patterns, etc.) than fully implantable systems. Additionally, the port-based system may also include additional connections (e.g., to deliver light and an electrical signal, in some instances chemicals/medications can be delivered through the port). One other value of a percutaneous system is that the percutaneous system can be used as a PBM trial for a period of time (e.g., as a trial), and if the trial proves successful, the patient might opt for a fully implantable system with an assurance that the full implantable system would work.

### III. Systems

An aspect of the present disclosure relates to systems that can provide chronic or temporary photobiomodulation (PBM) to many target areas. PBM generally refers to the delivery of a dose of light with a proper wavelength (e.g., one or more predefined wavelengths between 600 nm and 1200 nm) at a specific dosing scheme to a target area or target location within the body to achieve a desired non-thermal response (the response is assumed to be non-thermal). With PBM, different effects or responses can be achieved in different target areas or target locations, including nerve block, anti-inflammation (e.g., by activating anti-inflammatory microglia), anti-neurodegeneration (e.g., by overcoming cellular oxidative stress), anti-fibrotic responses in pathological fibrosis, improved cellular function (e.g., by improved cellular respiration), and the like.

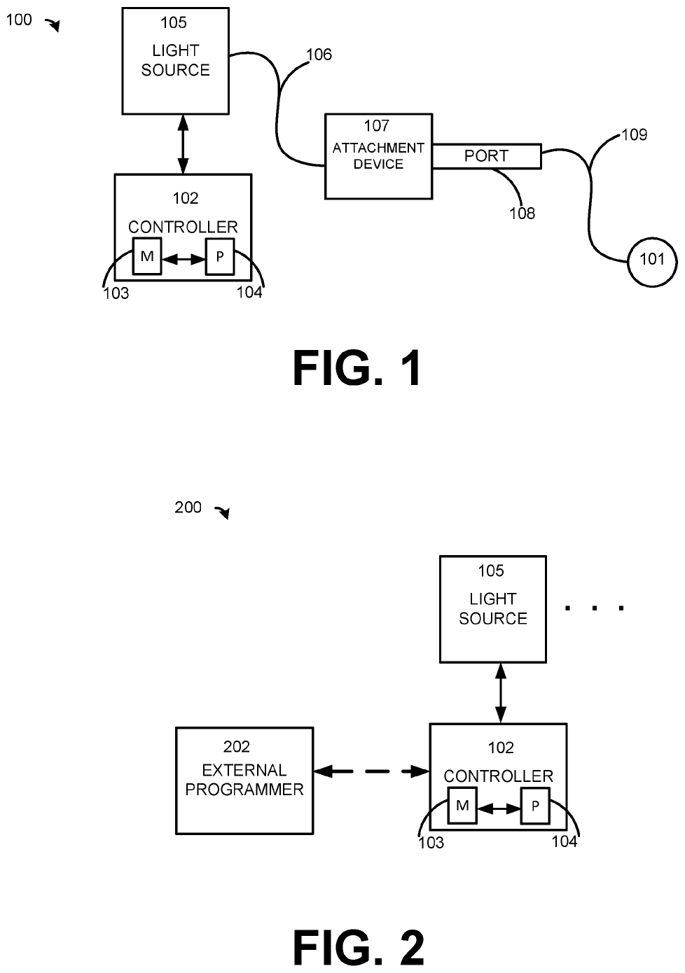

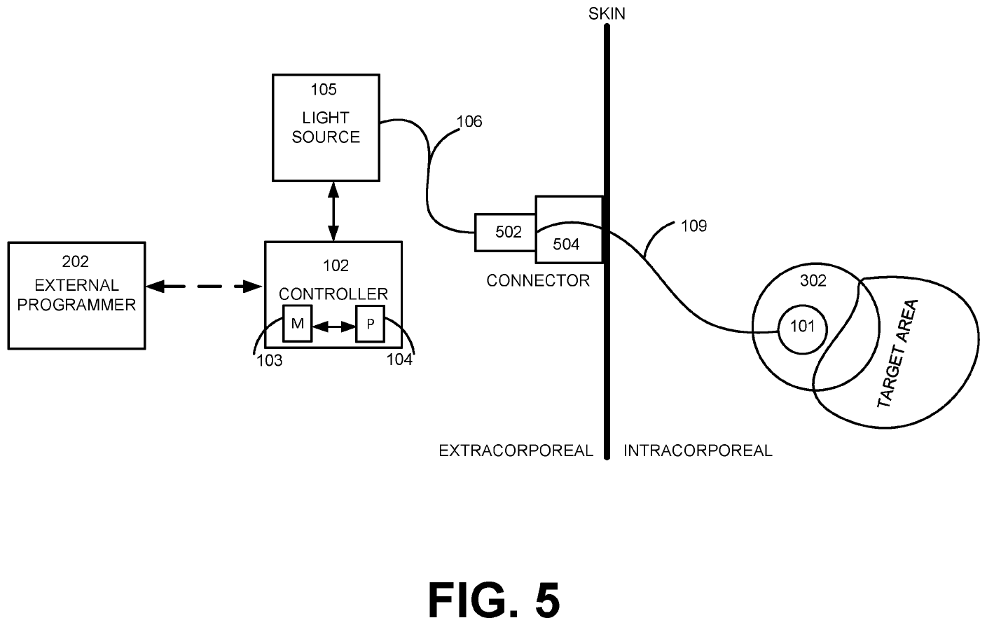

The systems can enter a patient's body and deliver the PBM to an associated target area percutaneously (through the skin from extracorporeal to intracorporeal). For example, a port can be one way to span the skin and provide an entrance from the extracorporeal region to the intracorporeal region, as shown, for example, in FIGS. 1-4 and/or 6-8. However, other devices can be used for percutaneous entry into the body (e.g., a light pipe itself without requiring a port), as shown, for example, in FIG. 5. It will be understood that the systems shown in FIGS. 1-8 are not mutually exclusive nor exhaustive of every different type of percutaneous delivery of light. FIGS. 1-8 are merely shown as examples of systems that can achieve percutaneous delivery of PBM. Modifications based on FIGS. 1-8 are covered by this disclosure.

As shown in FIG. 1, the PBM can be delivered to the target areas by a port-based system 100 (FIG. 1) that includes a controller 102 (that includes a memory (M) 103 and a processor (P) 104) in communication with a light source 105. The memory (M) 103 is one or more non-transitory devices that store data and instructions. The processor (P) 104 is a hardware device that accesses the memory (M) 103 and executes the instructions. It will be understood that in some instances, the functionality of the processor can be implemented in a microprocessor. In other instances, the processor can be implemented as a state machine or any other machine with processing ability. Although not illustrated, it will be understood that the controller can include additional hardware, such as a wireless transmitter that enables wireless communication with other devices, such as devices accessible within the cloud, devices associated with one or more clinicians, devices associated with the patient. In some instances, the controller 102 can be battery powered. In other instances, the controller 102 can receive line power. In still other instances, the controller 102 can recharge the battery via line power.

In the example shown in FIG. 1, the memory (M) 103 can store a predefined dosing requirement and the processor (P) 104 can access the memory (M) 103 and signal the light source 105 to generate a light signal for PBM of a target area within a patient's body based on the predefined dosing requirement. The predefined dosing requirement can include an optical power, a pulse width, a frequency, an intensity, a cycling parameter comprising one or more period(s) of on time or off time, an amount of light delivered per unit time, a total amount of light to be delivered, or the like. In some instances, like illustrated in FIG. 1, the controller 102 and the light source 105 are distinct devices. However, in other instances, the controller 102 and at least a portion of the light source 105 can be embodied in a common device. Additionally, in some instances, the light source 105 can include its own memory and/or processor. However, the light source 105 may be a slave to the controller and use the memory (M) 103 and/or processor (P) 104 of the controller. As an example, the light source 105 can be a laser, a laser diode, a light emitting diode, a broadband source, or the like that receives power either from its own power source or from a power source associated with the controller 102. The controller 102, in some instances, can have an associated on/off switch/button. In still other instances, the light source 105 can be integrated within an implanted portion of the port 108 (e.g., on either or both of the extracorporeal or the intracorporeal side).

The predefined dosing requirement can be programmed by a clinician using a clinician programmer. In some instances, the patient may not be able to change the dose configured by the clinician directly; instead, the clinician must perform the changes (e.g., during a clinic visit, a virtual visit, or over a network, like the cloud, or the like). In other instances, the patient may be able to change the dose to a different value, as long as the value is within a window (e.g., between a lower limit and an upper limit) that has been prescribed/preset by the clinician. It should be understood that in some instances, the clinician can program a certain number of PBM doses (like a prescription), after which the patient must return to the clinician for a new prescription. This would allow the clinician to evaluate the therapy for the patient, consider adjustments, and confirm system functionality. In other instances, the clinician can set an infinite number of doses. In still other instances, there can be a cap on the number of doses. In other instances, the predefined dosing requirement can be coupled to a feedback loop that decides the dosing.

The light source 105 can generate the light signal according to the predefined dosing requirement and send the light signal to an attachment device 107 (also referred to as port attachment device). In some instances, the light signal can be sent through a light pipe 106 (which may include an optical pipe and electrical wires). In other instances, the light source 105 can be directly connected to the attachment device 107, which can be directly connected to the port 108. Proper attachment of the attachment device 107 to the port 108 can be detected automatically such that the controller 102 will not power the light source 105 and/or deliver instructions to the light source 105 unless the connection is determined to be good. In some instances, the attachment device 107 can be connected to the port 108 using magnetic fields. In other instances, the attachment device 107 and the port 108 can have complementary mechanical features to facilitate the attachment and transmission of the light signal. Additionally, in some instances, the port 108 can have a unique identifier, such as an RFID, on an external portion of the port 108. The unique identifier can prevent a patient from using another patient's preprogrammed controller. In some instances, the port can provide a return signal indicative of light coupling.

The attachment device 107 can send the light signal to a port 108 to deliver the light signal to the target area within a patient's body. In other words, the attachment device 107 can facilitate transmission of the light signal from the light source 105 to the port 108. The attachment device 107 and the port 108 can have different configurations, but they always form a connection to deliver the light signal into the patient's body. The port 108 can be connected to a light pipe 109 to deliver the light signal to a light delivery element 101 to deliver the light signal to a target area or target location within the patient's body. In some instances, the controller 102 can have hardware that can determine if the external components (the light source 105, the light pipe 106, the attachment device 107, and the port 108) are connected properly. For example, the controller 102 can have/be in communication with a photosensor that can collect data related to light escaping from the external components.

As shown in FIG. 2, the controller 102 can be in wireless communication with an external programmer 202. Although illustrated as wireless communication, it will be understood that the controller 102 can engage in wired communication with the external programmer 202. The external programmer 202 can be one or more computing devices that may be remotely or locally located with respect to the controller 102. In some instances, the controller 102 and the external programmer 202 can be connected through the cloud and each can use the cloud to store data and instructions.

The external programmer 202 can provide or edit at least one aspect of the predefined dosing requirement used for PBM (e.g., dose parameters, total amount of light to be received by the patient, in a time, such as a day, week, month, 3 months, 6 months, 9 months, year, etc.). The external programmer 202 can also create a link between the controller 104, the light source 106, and/or the port 108. For example, the external programmer 202 can be a clinician programmer that resides in a clinician's office and can be used to set or edit the predefined dosing requirement, such as setting one or more optical dose parameters or defining a therapy program. The external programmer 202 can also receive communication from the controller 102 regarding progress of the patient using the PBM. For example, the controller 102 can track the amount of light that is or has been delivered to the patient over a period of time and this information can be transmitted to the external programmer 202. As an example, the therapy program can be stored in the cloud with a local copy stored in the memory (M) 103 of the controller so that the patient does not have to have the controller 102 connected to the internet to use the therapy program. As another example, the controller 102 can communicate with a device associated with the patient and convey pertinent information, such as the amount of therapy remaining on a prescription, the state of the batteries of the controller 102, illumination parameters, program usage data, or the like. In another example, the controller can receive data from a device associated with the patient including patient diary data, activity data, heart rate, physician indicated task, other health-related data, or the like. The controller as another example can receive data from sensing instrumentation that can be used to determine dosing. The controller 102 can aggregate the data in the cloud and make the data accessible to the external programmer 202.

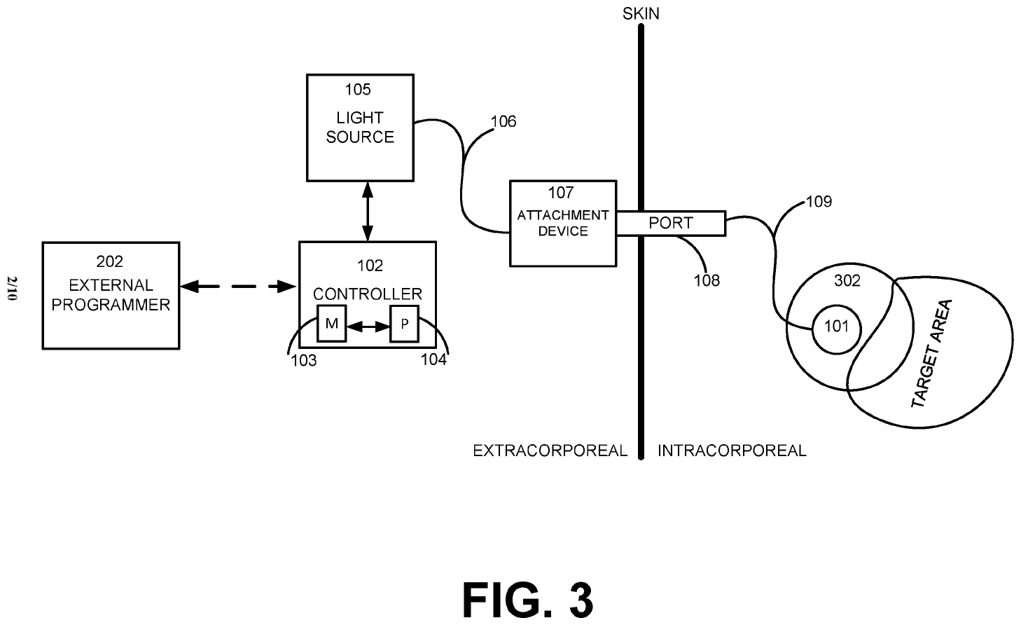

As shown in FIG. 3, the port 108 can span through a portion of the patient's skin, allowing light to travel into the patient's body. A portion of the port 108 (connected to the attachment device 107) can be within the extracorporeal portion of the system (in other words, outside the body). The controller 102, the external programmer 202, the light source 105, the light pipe 106, and the attachment device 107 can also be within the extracorporeal portion of the system. The light source 105 can be a standalone device, within the controller 102, within the attachment device 107, or within an external portion of the port 108. Another portion of the port 108 can be within the intracorporeal portion of the system (within the body). The light pipe 109 and the light delivery element 101 can also be within the intracorporeal portion of the system. The light delivery element 101 can deliver the light signal 302 to the target area within the patient's body.

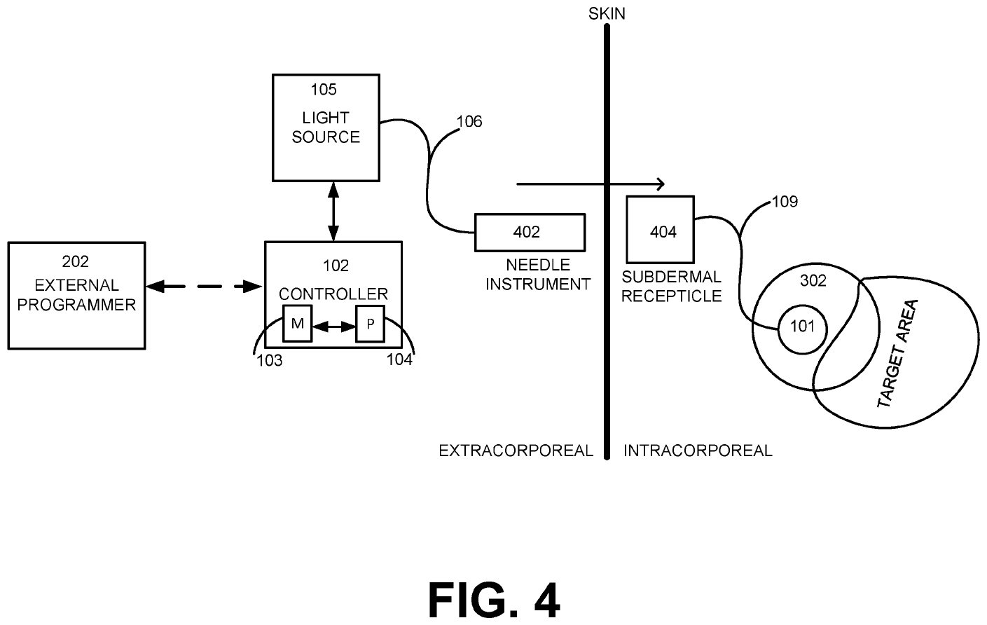

In some instances, light can be delivered through the skin to an implanted receptacle. One such example is shown in FIG. 4, where the attachment device can be a needle instrument 402 that is configured to pierce the patient's skin and travel from the extracorporeal region to the intracorporeal region. The needle instrument 402 can be a light-conveying needle instrument that can interface with a subdermal receptacle 404 that can transmit the light signal through the light pipe 109 to the light delivery element 101 for delivery to the target area. The subdermal receptacle 404 can be the probe. However, in this instance, the needle instrument is actually percutaneous.

Shown in FIG. 5 is an instance where the light pipe 109 is percutaneous. The light pipe 109 can transmit the light across/through the skin to the light delivery element 101 for delivery to the target area. The light pipe 109 can interface with a connector, which may consist of a temp (or removeable) connector 502 that interfaces with an external fixation device 504 that is fixed against the patient's skin (e.g., by sutures, staples, or other removeable fixation means) and provides a housing for the light pipe 109 leaving the body to hold the light pipe 109 in position. The system shown in FIG. 5 can be configured for temporary use as may be appropriate to address injuries or other situations that are not expected to be chronic (<1 year).

In some instances, the light pipe (e.g., both 106 and 109) can serve the functions of the port described above. The light pipe can act as the port with or without additional components like connector 502 and/or 504 and transmit light from the light source 105 to the light delivery element 101. For example, the light pipe can be secured to tissue internal to the dermal layers. As another example, the light pipe can be secured to dermal layers with tissue glue, sutures, suture sleeves, other device(s), other substance(s), or the like. In another example, the light pipe can include mechanical layers (e.g., clad, jacket, etc.) that make suitable fixation of the light pipe without damaging the fiber optic portion of the light pipe.

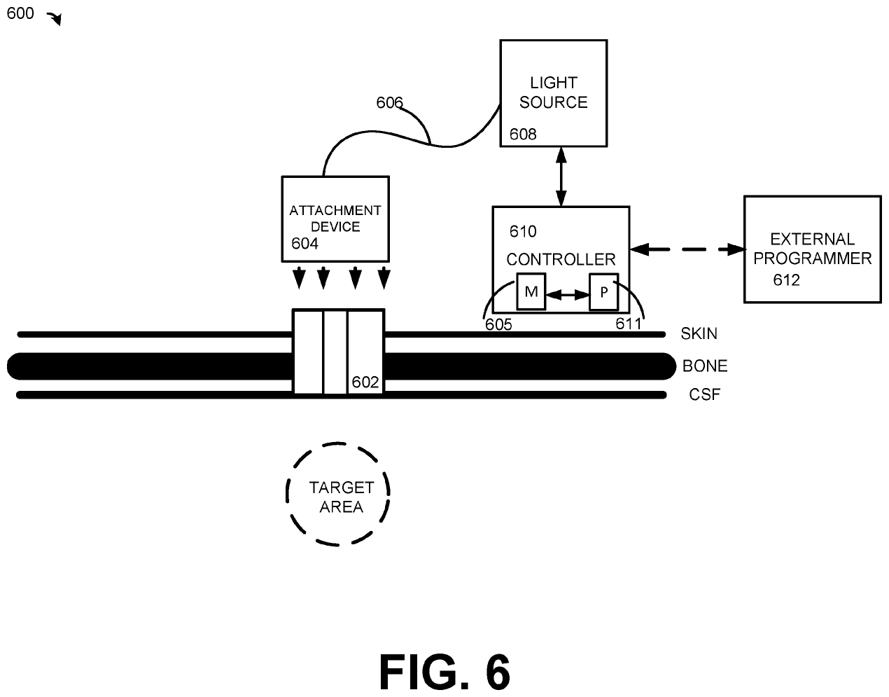

PBM can be delivered into the brain using the systems of FIGS. 6-8. The systems 600 and 700 can employ a port 602 (e.g., any of A, B, C, or D shown in FIG. 8) to deliver the light into the brain. In system 700, the port 602 is pre-existing because of electrodes 702a, 702b that are implanted in the brain. The port 602 can use the skull for attachment and may have additional head-specific concerns. It should be understood that two or more of the attachment device 604, the light transmission means 606, the light source 608, and the controller 610 (with the memory (M) 605 and the processor (P) 611) can be embodied in a single instrument. Moreover, the types of ports A, B, C, and D are not exclusive. The port can be attached to the skull by independent screws, press-fit, the whole device threaded, or the like. Additionally, each port can include the light source and the penetrating microelectrodes.

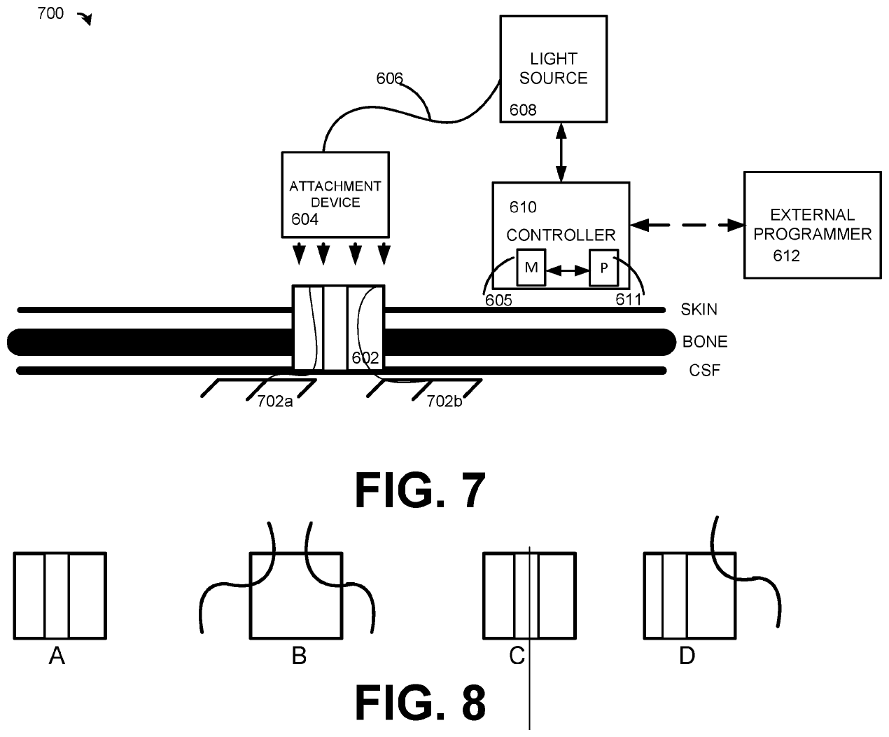

In some instances, the systems of FIGS. 6 and 7 can be configured for temporary use, which may be appropriate for treating an injury, but in other instances, the systems of FIGS. 6 and 7 can be configured for long-term (chronic or pseudo-chronic) use, which may be appropriate for treating a chronic disease. In instances of temporary use, the system can include a light pipe that enters into the brain (like C of FIG. 8). In instances of long-term use, the system can include different configurations, like A, B, or D of FIG. 8. Additionally, the microelectrodes 702a and 702b as shown in FIG. 7 can penetrate the cerebral cortex, a peripheral nerve, a peripheral ganglion, a tissue containing nerves or parts of nerves (e.g., an axon bundle), or the like. In some instances (not illustrated) the electrodes and the light source can share a common silicon substrate/wafer.

### IV. Methods

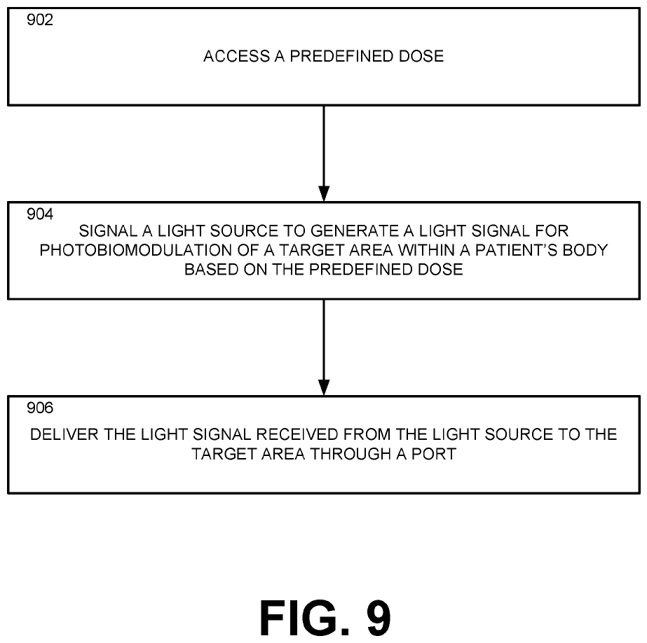

Another aspect of the present disclosure can include a method for using a system (shown in FIGS. 1-5, for example) for percutaneous photobiomodulation (PBM), as shown in FIG. 9. The method of FIG. 9 will be described with respect to the port-based system of FIGS. 1 and 2, but it will be understood that the systems of FIGS. 3-8 can be used similarly. In most basic form, the port-based system can include a controller 102 in communication (wired or wireless) with a light source 105 that generates a light signal that is delivered through the patient's skin to a target area (e.g., through components 106, 107, 108, and 109, with final delivery 101, for example). The controller 102 can also be in wireless communication with one or more external devices (e.g., external programmer 202). Steps of the method can be performed by the controller 102 that includes a memory storing a predefined dosing requirement 103 and a processor configured to access the memory and signal the light source to generate a light signal for PBM of a target area within a patient's body based on the predefined dosing requirement 104 (in FIGS. 1-5). As an example, the external programmer 202 can edit one or more aspects of the predefined dosing requirement.

For purposes of simplicity, the method is shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the method, nor is the method necessarily limited to the illustrated aspects. Additionally, at least the controller 102 is a computer-related entity that includes hardware, including a memory 103 (which is a non-transitory memory) and a processor 104, and communicates with hardware (e.g., light source 105 and external programmer 202) to facilitate the performance of port-based PBM.

At Step 902, a predefined dosing requirement (e.g., an optical power, a pulse width, a frequency, an intensity, a cycling parameter comprising a period of on time or off time, an amount of light delivered per unit time, a total amount of light to be delivered, etc.) can be accessed by the processor 104 of the controller 102. For example, the predefined dosing requirement can be stored in the memory 103 of the controller 102. The predefined dosing requirement can be received and/or edited based on instructions received wirelessly from a device associated with an external programmer 202.

At Step 904, a light source 105 can be signaled by the controller 102 to generate a light signal for PBM of a target area within a patient's body based on the predefined dosing requirement. The controller 102 can log information related to the light signal, including the number of doses given to the patient. For example, based on the number of doses given to the patient, the controller 102 can communicate wirelessly with a device associated with a doctor and the doctor can evaluate the usage.

At Step 906, the light signal received from the light source 105 can be delivered to the target area through a port (e.g., using elements 106, 107, 108, 109, and 101, for example). The light source 105 can be connected to the port 108 through an attachment device 107. The attachment device 107 can receive light from the light source (e.g., through external light pipe 106) and the port 108 can deliver the light into the body by the internal light pipe 109 and the light delivery element 101.

### V. Example Therapeutic Indications

The following therapeutic indications are described for context, but these therapeutic indications are not the only therapeutic indications treatable by the port-based systems described herein.

Treating Neuropathic and Nociceptive Pain: Pain can be treated temporally by pharmacological block of axons and cells that convey nociceptive information (small fibers, denoted C and Aδ). PBM has been shown to selectively block these same fibers at wavelengths in the neighborhood of 800 nm-840 nm at the proper doses, but other PBM wavelengths may also serve this purpose. Any sensory nerve or mixed (sensory & motor) nerve is then a target for PBM to treat pain. Example nerve targets to treat pain are (not to be limiting) are sciatic, saphenous, trigeminal, occipital, ulnar, radial, median, musculocutaneous, axillary, brachial plexus, inferior mesenteric, superior mesenteric, other nerves, and any branches of the aforementioned nerves.

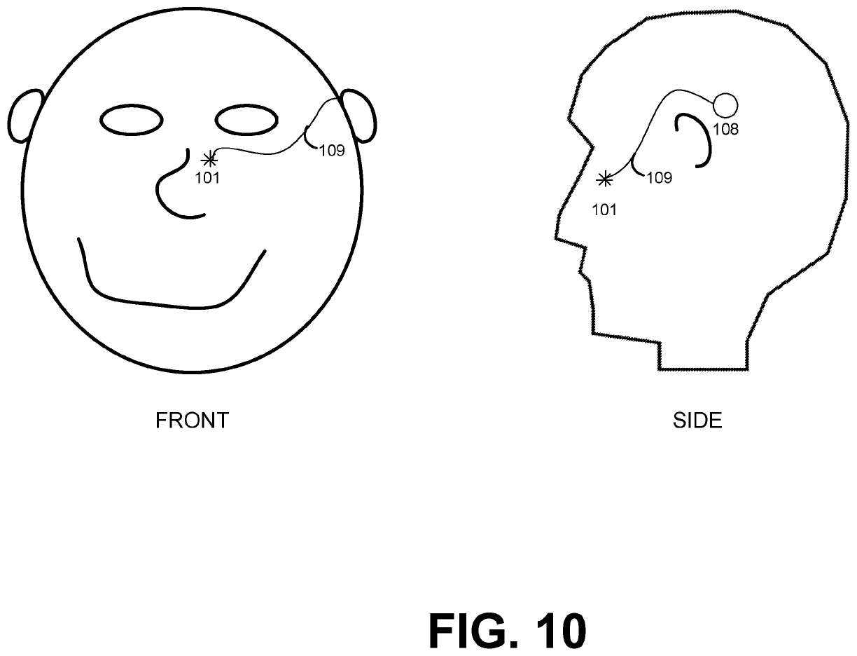

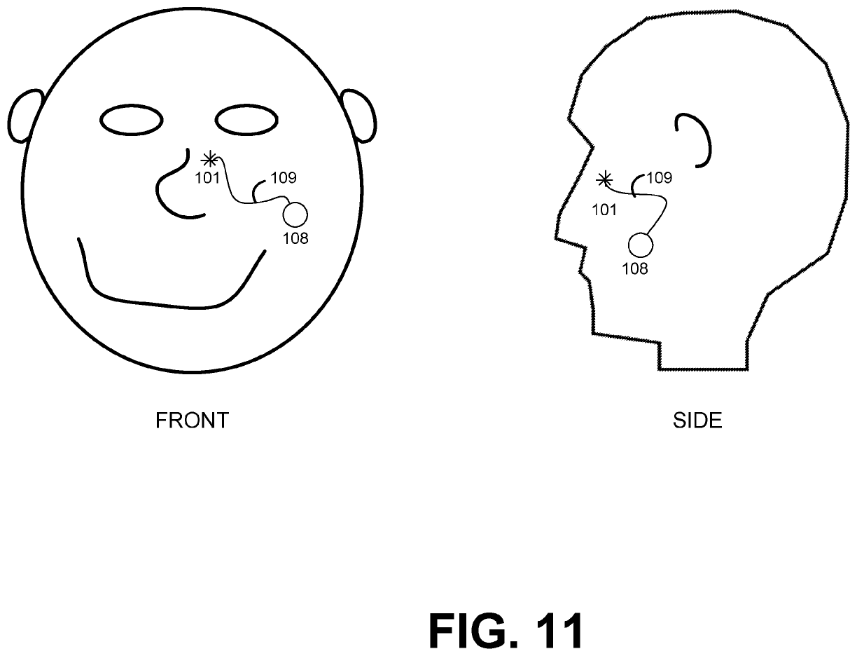

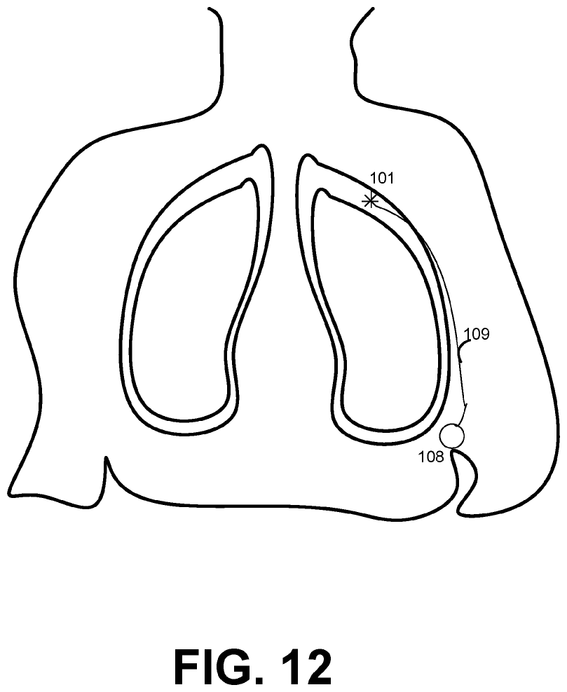

Block of the Sphenopalatine Ganglion (SPG): Pharmacological block of the SPG, composed of primarily small fibers, has been shown to effective treat headache. PBM of the SPG can be used for the treatment of headache syndromes (migraine, cluster, etc.). Different ways that the treatment can be applied percutaneously are shown in FIGS. 10 and 11.

Idiopathic Pulmonary Fibrosis (IPF): IPF is a serious condition where fibrosis encroaches on normal pulmonary tissue and breathing is progressively compromised until death a few years after diagnosis. Currently, no effective treatments exist. Data in a mouse model suggests that PBM can slow or inhibit the fibrosis progression. In human, light would need to be deliverable chronically. The treatment can be applied percutaneously as shown in FIG. 12.

Treating Maladies of the Brain: When the treatment region is the brain the port systems can use the skull for attachment and have other head-specific considerations. The maladies can be caused by stroke or traumatic brain injury (TBI). The maladies may also be due to neurodegenerative diseases, like Alzheimer's, Parkinson's, etc. The maladies might also be associated with inflammation, surgical trauma, such as resecting or cutting a tumor (e.g., on a nerve, on the spinal cord, on the brain, etc.), trauma subsequent to implanting a port, or trauma subsequent to implanting a device in the brain—such as a microelectrode array. The latter example is of interest because the trauma diminishes the effectiveness of the electrodes' ability to record neural signals. The port, in these instances, can span the skin and go through the subdermal area, the skill bone, and stop before or within the dura. In some instances, the port can be associated with one or more electrodes or microelectrodes. In other instances, the port can be associated with a light transmission pipe (like element 109 in any of FIGS. 3-5).

Treating Trauma Associated with Penetrating Microelectrodes: The act of placing electrodes in or on the brain, the spinal cord, on peripheral nerves, or the like causes inflammation and other effects that lead to neurodegeneration. This can cause the electrodes to become ineffective at sensing neural signals after only a temporary duration. Countering these effects is highly desirable. Further, patients with microelectrodes often have ports already for busing electrical signals, so adding light conveyance is a step using a foundation that exists for another purpose.

Light-induced Suppression of Activity in Nerves and Related Ganglia for Therapeutic Purposes: For example, light can be used for the following suppressive actions.
Suppression of small fiber afferent in the renal nerve and associated system to induce a renal deafferentation to treat hypertension. For example, hypertension may be caused by chronic kidney disease or other disease states, and treatable with renal deafferentation with PBM.
Suppression of small diameter afferents (e.g., C-fibers) in the glossopharyngeal nerve, superior laryngeal nerve (main branch and internal and external branches) and inferior laryngeal nerves to reduce or eliminate pain emanating from the upper airway including the tongue, larynx, and nasopharynx.
Suppression of small diameter afferents (e.g., C-fibers) in the superior laryngeal nerve (main branch and internal and external branches) and inferior laryngeal nerves to reduce or eliminate abnormal upper airway reactivity (e.g., excessive cough, reactivity to ingested substances and cold air) from the upper airway including the tongue, larynx, and nasopharynx.
Suppression of small diameter afferents (e.g., C-fibers) in recurrent laryngeal nerves to reduce or eliminate abnormal upper airway reactivity (e.g., excessive cough, reactivity to ingested substances and cold air) from the upper airway including the larynx and lower nasopharynx.
Suppression of small diameter afferents (e.g., C-fibers) in the glossopharyngeal nerve to reduce or eliminate upper airway obstruction caused by abnormal positioning of the tongue as well as closure of the nasopharynx—obstructive sleep apnea
Suppression of small diameter afferents (e.g., C-fibers) in the superior laryngeal nerve (main branch and internal and external branches) to eliminate upper airway obstruction caused by closure of the nasopharynx and larynx—obstructive sleep apnea
Suppression of small diameter afferents (e.g., C-fibers) in the inferior pharyngeal nerve to reduce or eliminate upper airway obstruction caused by closure of the nasopharynx—obstructive sleep apnea
Suppression of small diameter afferents (e.g., C-fibers) in the carotid sinus nerve to reduce or eliminate the development of hypertension and diabetes
Suppression of small diameter afferents (e.g., C-fibers) in the carotid sinus nerve to reduce or eliminate breathing disturbances associated with disease processes such as hypertension and diabetes
Suppression of small diameter afferents (e.g., C-fibers) in the cardiac nerve and/or the stellate ganglion to reduce or eliminate cardiac arrhythmias with disease processes such as congestive heart failure and pulmonary hypertension.

From the above description, those skilled in the art will perceive improvements, changes, and modifications. Such improvements, changes and modifications are within the skill of one in the art and are intended to be covered by the appended claims.

The following is claimed:

1. A system comprising:
a light source;
a controller comprising:
a memory storing a predefined dosing requirement;
a processor configured to access the memory and signal the light source to generate a light signal for photobiomodulation of a target area within a patient's body based on the predefined dosing requirement;
a port; and
an attachment device to deliver the light through a port, wherein the attachment device is configured to transmit the light signal from the light source through the port for transmission to the target area.

2. The system of claim 1, wherein the light source, the controller and the attachment device are each external to the patient's body.

3. The system of claim 2, wherein the light source is within the controller, the attachment device, or a portion of the port.

4. The system of claim 1, wherein the port is configured to span a portion of skin of a patient.

5. The system of claim 1, wherein the predefined dosing requirement comprises an optical power and at least one of a pulse width, a frequency, an intensity, and a cycling parameter comprising a period of on time or off time.

6. The system of claim 1, wherein the predefined dosing requirement comprises an amount of light delivered per unit time and/or a total amount of light to be delivered.

7. The system of claim 1, wherein the port attachment device comprises an external light pipe extending from the light source controller to the port.

8. The system of claim 1, wherein the port attachment device comprises a light-conveying needle instrument.

9. The system of claim 8, wherein the port is configured to be implantable as a subdermal receptacle.

10. The system of claim 1, wherein the port is connectable to an implantable light pipe that extends from the port to the target tissue.

11. The system of claim 10, wherein the implantable light pipe is configured to deliver the predefined dosing requirement of the light signal from the port to the target area.

12. The system of claim 10, wherein the port is configured to be external to the patient's body and the implantable light pipe is configured to span through the patient's skin.

13. The system of claim 12, wherein the port comprises a fixation connector configured to connect to the implantable light probe and a connector configured to connect to the external light pipe.

14. The system of claim 1, wherein the light source comprises at least one of a laser diode or a light emitting diode.

15. The system of claim 1, wherein the controller further comprises a wireless transmitter to communicate with the cloud, a device associated with a clinician, or a device associated with the patient.

16. A method comprising:
accessing, by a controller comprising a processor, a predefined dosing requirement;
signaling, by the controller, a light source to generate a light signal for photobiomodulation of a target area within a patient's body based on the predefined dosing requirement;
delivering the light signal received from the light source to the target area through a port,
wherein the light source and the port are connected through an attachment device.

17. The method of claim 16, further comprising receiving, by the controller, the predefined dosing requirement from a clinician.

18. The method of claim 16, wherein the predefined dosing requirement comprises an optical power and at least one of a pulse width, a frequency, a pulse shape, an intensity, and a cycling parameter comprising a period of on time or off time.

19. The method of claim 16, wherein the predefined dosing requirement comprises an amount of light delivered per unit time and/or a total amount of light to be delivered.

20. The method of claim 16, further comprising logging, by the controller, a number of doses given to the patient.
